(19) United States
(12) Patent Application Publication
(10) Pub. No.: US 2009/0203023 A1
(43) Pub. Date: Aug. 13, 2009

(54) Concentrating Microorganisms in Aqueous Solution Prior to Selective Staining and Detection

(75) Inventor: Paul E. Johnson, Laramie, WY (US)

Correspondence Address: JENNIFER L. BALES, MOUNTAIN VIEW PLAZA, 1520 EUCLID CIRCLE, LAFAYETTE, CO, 80026-1250, US

(73) Assignee: UNIVERSITY OF WYOMING, Laramie, WY (US)

(21) Appl. No.: 12/369,576

(22) Filed: Feb. 11, 2009

Related U.S. Application Data
(60) Provisional application No. 61/065,340, filed on Feb. 11, 2008.

(51) Int. Cl. C12Q 1/68; C12Q 1/02; C12M 1/34

(52) U.S. Cl. 435/6; 435/29; 435/288.7

(57) ABSTRACT

Apparatus and methods for detecting a small number of target microorganisms found in a large volume of aqueous solution by diverting small volumes containing fluorescent microorganisms, including target microorganisms, into a smaller specimen of solution, followed by selectively staining the specimen to tag the target microorganisms and detecting the tagged target microorganisms.

This application claims priority to provisional application for patent No. 61/065,340 filed Feb. 11, 2008.

### BACKGROUND

1. Field of the Invention

The present invention relates to apparatus and methods for concentrating small numbers of target microorganisms found in a large volume of aqueous solution into a smaller volume of solution, followed by selective staining and detection of the target microorganisms.

2. Description of the Related Art

The present inventor has developed techniques and devices for sensitively detecting potentially pathogenic microorganisms (including bacteria, fungi, and protozoa) using Fountain Flow™ cytometry in transparent or translucent fluids. FIGS. 1-3 (Prior art) illustrate these techniques, which are described in U.S. Pat. Nos. 6,765,656 and 7,161,665, as well as U.S. patent application Ser. Nos. 11/328,003 and 12/214,549, all to the present inventor and incorporated by reference herein.

For example, U.S. Pat. No. 6,765,656 to the present inventor teaches a Fountain Flow™ Cytometer, wherein a sample of fluorescently tagged cells flows up a tube (for example in a flow cell) toward a CCD camera and foreoptics. FIG. 1 (Prior Art) shows such a system. FIG. 2 shows the flow cell in more detail. FIG. 3 shows another embodiment of such a system, with an LED illuminator and a CMOS camera. The cells are illuminated in the focal plane by a laser or LED through a transparent end element. When the cell(s) pass through the camera focal plane, they are imaged by a CCD or CMOS camera and a lens assembly, through a transparent window and a filter that isolates the wavelength of fluorescent emission. The fluid in which the cells are suspended then passes by the window and out the effluent tubing.

There are many and various clinical and industrial applications requiring the detection and/or enumeration of microorganisms in various background matrices (e.g. food, beverages, and body fluids) for quality control and clinical diagnoses. It is common practice to use a dye specific to certain types of microorganism so that it is easy to contrast cells of interest against a background of other particles, including other kinds of cells/microorganisms. This is particularly important in techniques such as Fountain Flow™ cytometry or conventional flow cytometry whereby an automated method is used to detect and count cells of interest based on their fluorescent intensity. In these cases it is common to use one of a variety of stains that is specific to the microorganism of interest, for example an immunolabel, where fluorescent molecules are attached to an antibody specific to the microorganism of interest. However, there are applications where detections of smaller numbers of selected microorganisms in large volumes of water are cost-prohibitive owing to the large volume of expensive dye necessary (e.g. antibody dyes and fluorescent in situ hybridization dyes). With this in mind, it is desirable to produce a method that separates out a smaller volume of fluid containing most of the microorganisms of interest as well as other microorganisms, which may then be dyed with a more expensive dye which labels target microorganisms.

### SUMMARY

An object of the present invention is to provide apparatus and methods for identifying target microorganisms in a fluid sample. A system according to the present invention first separates out a fraction of the fluid sample containing most of the target microorganisms and then dyes that fraction with a dye specific to the target organisms.

The process is started with a fluid sample containing fluorescent particles (target and non-target). If the particles are not naturally fluorescent, a fluorescent dye may be used to fluoresce them. The separation step is accomplished by passing the sample through a fluorescent detection device. When the detection device detects a fluorescent particle, it temporarily diverts the sample flow exiting the detector into a specimen reservoir. The rest of the time, the sample flow exiting the detector flows into a waste receptacle, such as a reservoir or a drain. The particles that are detected and diverted will generally include most of the target particles, but will also generally include many other particles that also exhibit fluorescence. Whether the target particles are naturally fluorescent, or are dyed with a fluorescent dye, other similar particles will also be false positives during this phase of the process.

The sample portion in the specimen reservoir thus contains a higher concentration of target particles (and non-target particles). The next phase of the process comprises labeling the target particles with a dye specific to the target particles. This allows the target particles to be detected and counted.

### BRIEF DESCRIPTION OF THE DRAWINGS

FIG. 1 (Prior Art) is a side schematic view of a device for detecting fluorescently tagged cells using a flow cell with a laser illuminator and a CCD camera.

FIG. 2 (Prior Art) is a detailed side cutaway view of the flow cell of FIG. 1.

FIG. 3 (Prior Art) is a side schematic view of a device for detecting fluorescently tagged cells using a flow cell with an LED illuminator and a CMOS camera.

FIG. 4 is a schematic drawing showing an embodiment of the apparatus for separating out a fraction of a fluid sample containing most of the target microorganisms into a specimen reservoir.

FIG. 5 is a flow diagram illustrating a process of separation accomplished by the apparatus of FIG. 4.

FIG. 6 is a schematic drawing illustrating an embodiment of the apparatus for detecting and counting target particles within the specimen reservoir.

FIGS. 7A, 7B, and 7C are schematic drawings illustrating other embodiments of apparatus for detecting and counting target particles within the specimen reservoir.

FIG. 8A is a plan view of an improved flow cell for use in the separating apparatus. FIG. 8B is a side cutaway of the improved flow cell.

FIG. 9 is a plot illustrating the ability of the present invention to detect algae based on their natural fluorescence.

### DETAILED DESCRIPTION OF THE PREFERRED EMBODIMENTS

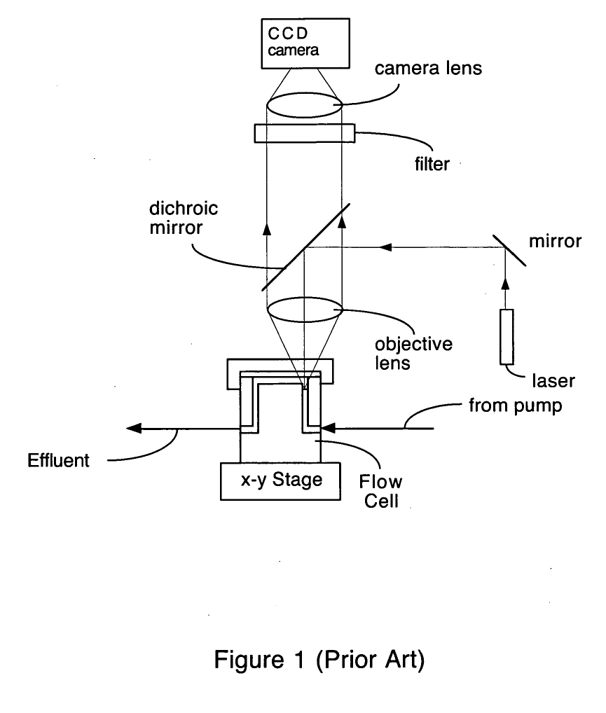

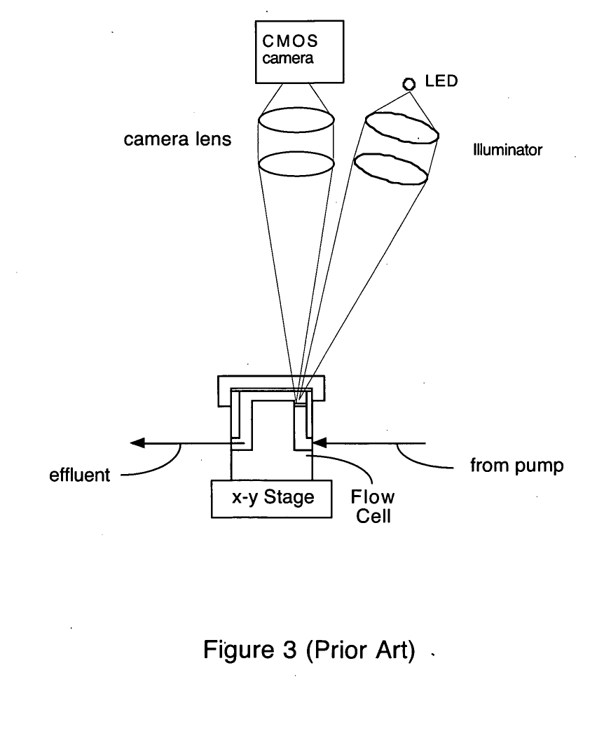

The present invention allows fluorescence measurements of specific microorganisms in a fluid sample, in particular blood, blood products, freshwater, and saltwater. These are applications where detection of smaller numbers of target microorganisms in large volumes of water is cost-prohibitive, owing to the large volume of expensive dye necessary to specifically tag the target microorganisms (e.g. antibody dyes and fluorescent in situ hybridization dyes). The process utilizes a less expensive dye (or natural fluorescence) to separate out a smaller volume of specimen containing most of the microorganisms of interest (as well as other microorganisms present). The smaller volume is then dyed with a more expensive dye specific to the target microorganisms and then examined for target microorganisms. The relatively inexpensive dye separates cells from the aqueous sample that include, as a subset, target cells of interest. The invention may use a Fountain Flow™ cytometer (refer to FIGS. 1-3, Prior Art) to identify fluorescing cells. In this case, the process may be designated Fountain Flow™ Sorting (FFS). Detection of fluorescing cells activates, for example, a solenoid-driven nozzle that can separate cells into one of two receptacles.

The embodiments below illustrate examples of apparatus and methods for enumerating target microorganisms found in small numbers in an aqueous solution. First, separation is performed in order to concentrate microorganisms, as well as some non-target microorganisms and detritus, into a much smaller specimen. Then, target microorganisms are specifically tagged and detected/enumerated.

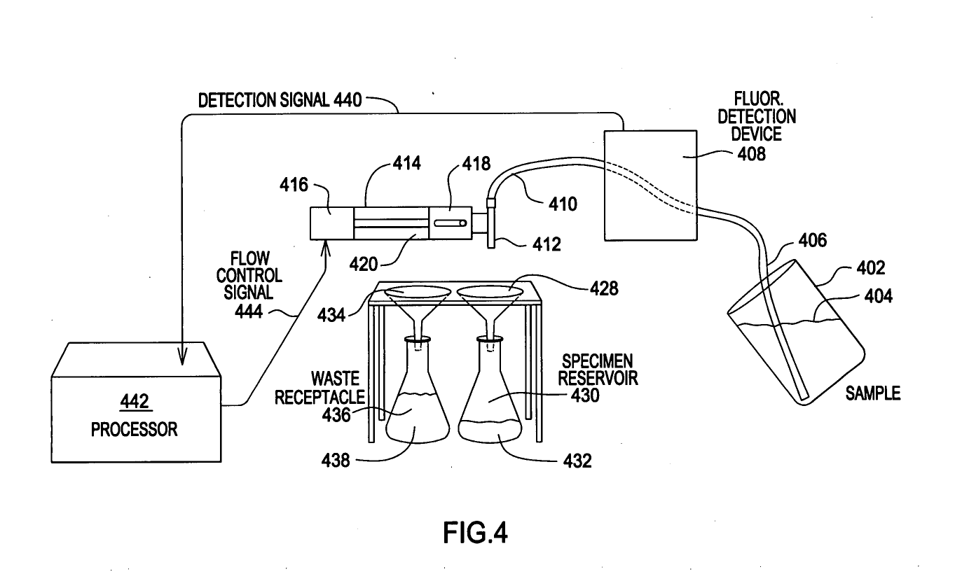

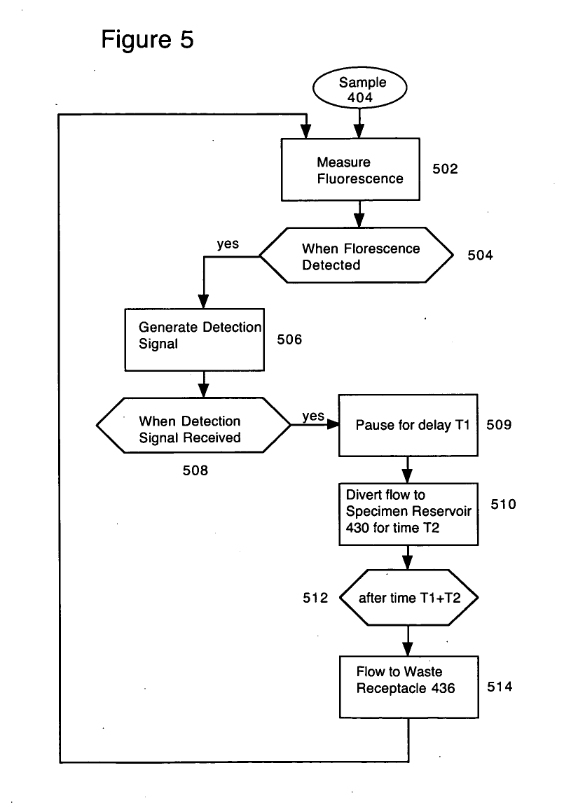

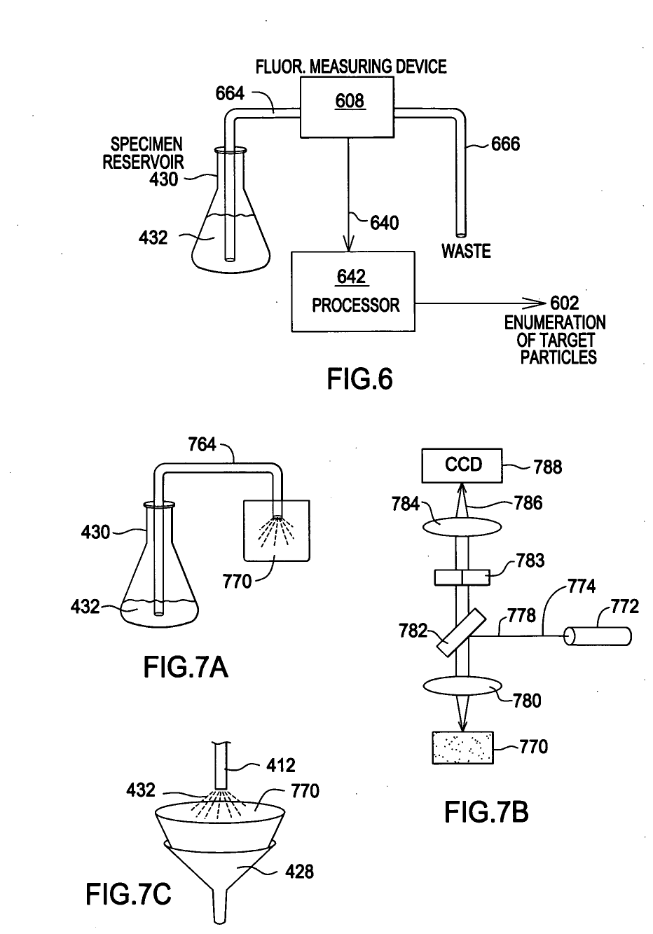

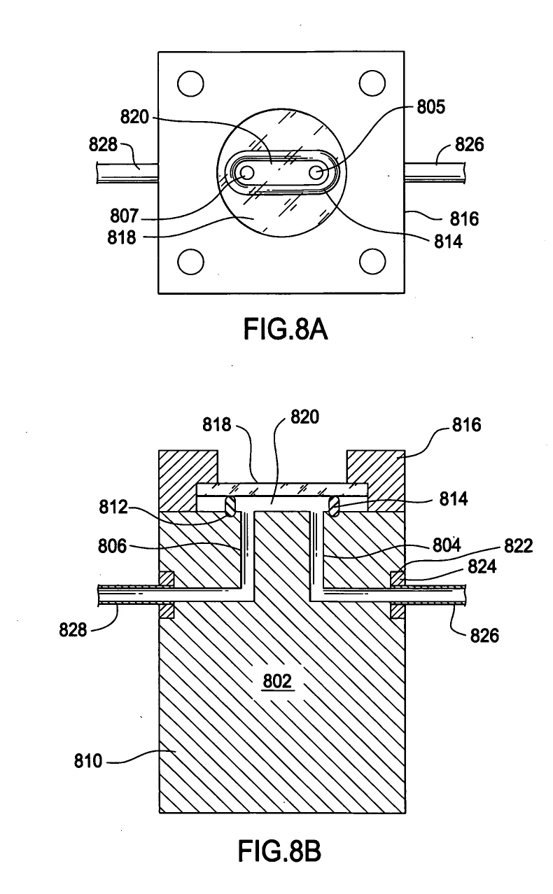

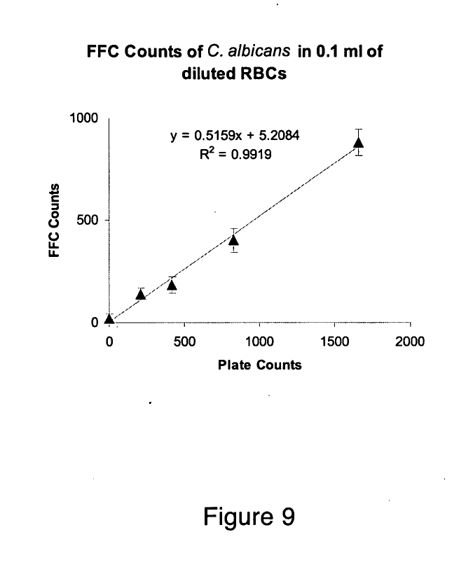

FIGS. 4, 6, and 7A-7C show examples of apparatus used in the separation and enumerating processes. FIG. 5 shows a process performed in the separation step. FIGS. 6 and 7A-7C show apparatus for measuring target particles. FIGS. 8A and 8B show an improved flow cell for use in the present invention, and FIG. 9 illustrates the performance of the invention.

FIG. 4 is a schematic drawing showing an embodiment of the apparatus for separating out portions of a fluid sample 404 containing most of the target microorganisms into a specimen reservoir 430.

Sample 404 from container 402 is generally an aqueous solution containing a low concentration of target microorganisms, as well as other, non-target microorganisms. These target microorganisms, as well as some non-target microorganisms and detritus (i.e. false positive detections), will be detected by their fluorescence. Some microorganisms are naturally fluorescent, while others require fluorescent tagging with a dye.

Sample 404 is flowed through fluorescent detection device 408 via tubing 406. Fluorescent detection device 408 detects the fluorescent particles and generates a fluorescence detection signal 440. Detection signal 440 is used by processor 442 to generate a flow control signal 444. Flow control signal 444 causes flow control element 414 to divert the sample issuing from detection device 408, via tubing 410 and spout 412, into specimen receptacle 430 for a period after a fluorescent particle is detected. Generally, a small volume of sample containing a fluorescent particle is diverted into specimen reservoir 430, before flow returns to waste receptacle 436. Thus, a fraction of the sample containing most of the target particles (as well as some non-target particles) becomes specimen 432.

The rest of the time, the sample issuing from detection device 408 flows into waste reservoir 436, becoming waste 438 which can generally be discarded.

An example of a flow control element 414 is shown in FIG. 4. In this example a solenoid 416 causes a rod 420 to move to the right, causing spout 412 to be disposed above funnel 428 and sample to flow into specimen reservoir 430. Solenoid 416 is triggered by flow control signal 444. After a short period, solenoid 416 releases, and spout 412 moves to the left and disposes over funnel 434, causing sample to flow into waste receptacle 436. A bracket 418 controls the slide axis of rod 420. A biasing element such as a spring (not shown) may be used to bias the spout into a default position (over funnel 428 or funnel 434) when solenoid 416 is not operating, while solenoid 416 moves spout 412 into the non-default position.

Preferably, only a small portion of sample is diverted into specimen reservoir 430 (for example on the order of 1% of the entire sample) so that an expensive dye specific to the target microorganisms can be used sparingly.

In a preferred embodiment, an inexpensive dye, e.g. SYBR Green (Invitrogen, Eugene, Oreg.), is mixed with a relatively large volume of sample 404 (10 ml to 1 liter). After a sufficient period of time has elapsed (usually a few minutes or even less), the sample is run through fluorescence detector 408 (e.g. a Fountain Flow™ cytometer), the output of which flows to flow control element 414 which causes spout 412 to empty into either specimen reservoir 430 or waste receptacle 436. When no fluorescent particles are detected by the Fountain Flow™ cytometer, the flow empties into waste receptacle 436, and when a fluorescent particle is detected, solenoid 416 drives spout 412 over to specimen reservoir 430, which thus collects fluorescent organisms above some threshold brightness. The solenoid can be controlled through an interface by the same processor 442 that is used to detect microorganisms in the Fountain Flow™ cytometry system. For rare cell detection with minimal false positives, specimen reservoir 430 will contain a relatively small amount of liquid specimen 432, which can be stained with a more selective, more expensive secondary stain selected to tag the target particles. Then the particles in specimen reservoir 430 can be measured by epifluorescence microscopy, conventional flow cytometry, Fountain Flow™ cytometry, etc. Examples of this process are shown in FIGS. 6-7B.

It is preferable that there is not a great deal of dead fluid volume between detection and nozzle 412. If the velocity of particles through the detection device 408 and flow control element 414 were absolutely constant, then it would be possible to position the spout over specimen reservoir 430 for precisely the amount of time necessary to place an extremely small liquid volume containing the detected particle. However, particles will vary in velocity according to their trajectory and orientation in the flow cell and sorter. The optimum means of collecting small volumes of water in specimen reservoir 430 while still collecting most of the detected particles, is to minimize the volume between the detection device 408 and sorter spout 412. These design constraints are discussed in more detail in conjunction with FIGS. 8A-B and 9.

FIG. 5 is a flow diagram illustrating an example of a process of separation accomplished by the apparatus of FIG. 4. In step 502, fluorescence detector 408 measures fluorescence within sample 404, which contains fluorescent target and non-target particles. When fluorescence is detected 504, a detection signal 440 is generated in step 506. Processor 442 receives the detection signal in step 508, and generates a flow control signal 444. In response, flow control element 414 first waits a period T1 in step 509, and then diverts the sample flow to specimen reservoir for a period T2 in step 510. After period T1 plus T2 passes in step 512, flow returns to the waste receptacle 436 in step 514. Periods T1 and T2 are selected to result in a target particle being sorted into specimen reservoir 430 with a given probability, for example >75%. Periods T1 and T2 could be selected periods of time, or could be determined by flow volume.

As an alternative, fluorescence detection signal 440 could function as the flow control signal, removing the necessity of generating the flow control signal separately.

When the concentration of target microorganisms is small, it may not be necessary to be concerned about a second target microorganism passing through the system during T1 or T2. It may sometimes be desirable to continue diverting the sample if a second target microorganism is detected during T2, or to lengthen the upcoming T2 if a second target microorganism is detected during T1.

FIG. 6 is a schematic drawing illustrating an embodiment of the apparatus for detecting and counting target particles within the specimen 432 in specimen reservoir 430. The target particles in specimen 432 are tagged using a dye specific to the target organism. In the embodiment of FIG. 6, specimen 432 is flowed through a fluorescence detection device 608 (for example by a pump, not shown) via tube 664. When detection device 608 detects fluorescence from the tagged target particles, a detection signal 640 is generated. Processor 642 counts the detection signals and thus enumerates 602 the target particles in specimen 432. All of the specimen may be examined, or only a representative portion.

FIGS. 7A-7C are schematic drawings illustrating further embodiments of apparatus for detecting and counting target particles in specimen 432. Again the target particles have been tagged.

Specimen 432 is filtered through filter 770 in FIG. 7A, after specimen 432 has been collected in specimen reservoir 430. Specimen 432 may be pumped via tube 764 from reservoir 430 as shown in FIG. 7A (pump not shown) or specimen 432 could be removed by a syringe or the like and filtered through filter 770. As another alternative, a portion of the specimen could be inserted into a counting chamber with ˜100 μl volume, or into chamber slide (such as a Nageotte slide), and the chamber slide inspected using an epifluorescence microscope or the like.

Note that in some embodiments it is desirable to concentrate the target particles into a small area on the filter 770, say a few mm on a side, in order to have the particles concentrated into a single microscope field of view. In this case, the output of 764 would be a fine stream rather than a spray as shown.

In FIG. 7B, filter 770 (or chamber slide, etc., not shown) is examined to count the fluorescent target particles on filter 770. The example epifluorescence microscope of FIG. 7B includes an illumination source 772 such as one or more lasers generating light beams 774, 778; a dichroic mirror 782 for directing beams 774, 778 onto filter 770 and for directing light 786 reflected from filter 770 toward CCD 788; optics 780 and 784; and filter 783. CCD (or other detector) 788 detects fluorescent particles on filter 770 (or other specimen). Epifluorescence microscopes have a variety of configurations that may be used.

In the embodiment of FIG. 7C, specimen 432 is filtered as it is collected, into filter 770 disposed within funnel 428. In the embodiment of FIG. 7C, the specimen reservoir would actually be the filter 770, and the dye for the target particles would be applied to the filter. As an alternative, filter 770 could be disposed beneath funnel 428.

Below, two examples of target microorganism enumeration according to the present invention are described.

### 1. Use of Fountain Flow™ Sorting (FFS) for Coastal Monitoring of Seawater for Toxic Algae

One application the present invention is to provide an inexpensive, portable, and rapid means for the detection of toxic algae in seawater. Such use can lessen the public health and economic impact of toxic algal blooms, especially in coastal communities, which depend on harvesting fish and shellfish. Early detection will allow steps for mitigation to be put into place to prevent large economic losses, such as the early transfer of infected shellfish to decontamination tanks. Currently available techniques do not allow for the detection of toxic algae in a sufficiently timely way.

FFS (Fountain Flow™Sorting) performs detection and species identification of algae at very low concentrations (<50/liter), which allows sorting algal cells from a large (100-500-ml) sample, based on their autofluorescence (from chlorophyll and other natural fluorochromes found in algae), into a much smaller (˜100-200 μl) subsample that is then stained with a relatively expensive dye for species identification.

One dye of choice would be custom rRNA FISH (Fluorescent in Situ Hybridization) probes specific to the toxic algal species of interest (as described by Metfies et al. in 2006). Peptide nucleic acid (PNA) FISH uses a synthetic fluorescent molecule that binds only to a specific rRNA sequence, designed to identify a specific toxic species.

In one embodiment, specimen 432 is filtered onto a black polycarbonate filter 770, stained with an rRNA FISH dye, and enumerated with an epifluorescence microscope 700 using emission/illumination filters 776, 784, optimized for that dye. In another embodiment the subsample is filtered onto black polycarbonate filter and inserted into a handheld imaging/counting device that performs the counting automatically. As another alternative, The Countess™ from Invitrogen uses a chamber slide, so a portion of the specimen may be examined without filtering. Note that direct filtration/labeling of the primary sample 404 is not practical owing to clogging of filters when significant volumes of seawater are sampled. Filtration results in clogged filters when significant volumes of seawater are sampled.

In this embodiment samples 404 are examined for toxic algae in a multiple-step process: 1) The 100-ml to 1-liter seawater samples are sorted by autofluorescence intensity (at the optimal wavelength bandpasses for chlorophyll a and b). 2) seawater samples 404 are flowed through the Fountain Flow™ Sorter in order to separate (>75%) algal cells into a smaller (˜100 μml) volume (at flow rates of 5-100 ml per minute), which are be filtered onto a polycarbonate (for example) filter 770, 3) algae on the filter will be stained with one or both of the two rRNA FISH probes specific to the two species (for example) of toxic algae being tested, 4) the filter will be examined by direct epifluorescence microscopy 700 for species identification and enumeration.

In two alternate embodiments step 4 is replaced by enumeration using a portable optical (imaging) counter, such as the Countess™ from Invitrogen, or the sample is passed through a Fountain Flow™ cytometer a second time after step 3 above.

### 2. Use of Fountain Flow™ Sorting for the Detection of Fungi in Human Blood

The invention described here can be used for the detection of microorganisms in blood and blood products. A family of embodiments can be used for the detection of the Candida fungus in human blood.

Timely and appropriate antibiotic treatment for sepsis has been shown to be critical, significantly decreasing mortality (MacArthur et al., 2004). It has been shown (Kumar et al., 2006) that there is a 7% rise per hour, after the onset of septic shock, in the mortality rate from untreated sepsis in general. Culture-based detection systems represent the current accepted practice in the detection of bacterial/fungal bloodstream infection, but take days to complete.

One particular problem with treatment of fungemia is the high incidence of Candidemia caused by Candida glabrata, which is resistant to fluconazole, the most common drug used to treat Candidemia (Kauffman, 2005). The “Guidelines for Treatment of Candidiasis” (Pappas et al., 2004) recommends that identification of the infecting fungus should be used to guide therapy. They specifically recommend fluconazole for C. albicans (without prior exposure to azole), while other antifungal drugs should be used for C. glabrata. The difference in efficacy, toxicity, and expense of differing drug therapies is important. Thus, any rapid technique or suite of techniques that can identify to the species level are key. The potential cost savings from using appropriate drug therapy in Candidemia patients has been estimated at $1,808 per patient (Forrest et al., 2006).

The embodiment of the invention describe here is to Candidemia diagnosis in septic patients. FFS detects, enumerates and identifies the species of Candida at very low concentrations (<5/ml), which (as above) allows us to sort fungal cells, stained with an inexpensive dye, from a large (10-ml) sample 404 into a much smaller (e.g. 100-μl) specimen 432 that is then stained with more-expensive dyes for species identification. As antibodies for strains of Candida other than Candida albicans are not commercially available, this embodiment would include the use Fluorescent In Situ Hybridization (FISH) dyes for direct microscopic identification once a positive detection is made of Candida in blood (Forrest et al., 2006; Trnovsky et al., 2008). FISH uses a synthetic fluorescent molecule that binds only to a specific rRNA sequence, designed to identify a specific species.

Samples are be examined for Candida in a multiple-step process: 1) The 5-10 ml samples 404 are incubated with a fluorescent fungal cell wall (chitin/cellulose) dye (such as Calcofluor White, Pontamine Fast Scarlet, or Solophenyl Flavine) with reagents to suppress fluorescence from background (especially leukocytes), 2) the stained sample is flowed through the Fountain Flow™ Sorter 408 to separate (>75%) yeast cells into a smaller (˜100 μl) volume specimen 432, which is then filtered onto a polycarbonate filter 770, 3) Candida on the filter is stained with a PNA (peptide nucleic acid) FISH dye specific to one (or more) species of Candida, and 4) the filter is examined by direct epifluorescence microscopy 700 for species identification and enumeration. The PNA FISH dyes are commercially available from AdvanDx (Woburn, Mass.).

The inventor has developed a dye combination to minimize false-positive detections for the primary (pre-sort) detection and has submitted a patent application for this technology (U.S. patent application Ser. No. 12/214,549, incorporated herein by reference).

In this embodiment one could use a PNA FISH assay sold by AdvanDx for the secondary, post-sort, species identification and enumeration. This FDA-approved assay, which differentiates Candida albicans and Candida glabrata, is commonly used to label one cultured colony or 20 μl of liquid culture, and it is cost-effective for identification at these small levels of sample, but it is not viable for detection of low concentrations of Candida in blood (<100 cells/ml), as required for early and rapid detection of Candidemia.

In the standard AdvanDx protocol, 20 μl of liquid culture is placed in a microcentrifuge tube with 0.2 ml of PNA combined fixing/hybridization reagent for Candida albicans and Candida glabrata. (The C. albicans FISH is used with a green FITC fluorochrome, and the C. glabrata FISH is used with a red fluorochrome). The contents are vortexed, incubated at 55 C for 30 min., centrifuged, and washed twice (incubated at 55 C in the wash buffer for 10 minutes each time), and then mounted on a glass slide, dried, and examined by epifluorescence microscope. In the present embodiment, cells are filtered onto a polycarbonate filter 770 for microscopic examination, either before or after FISH staining. This process is executed in samples for which yeast has already been detected using the primary stain.

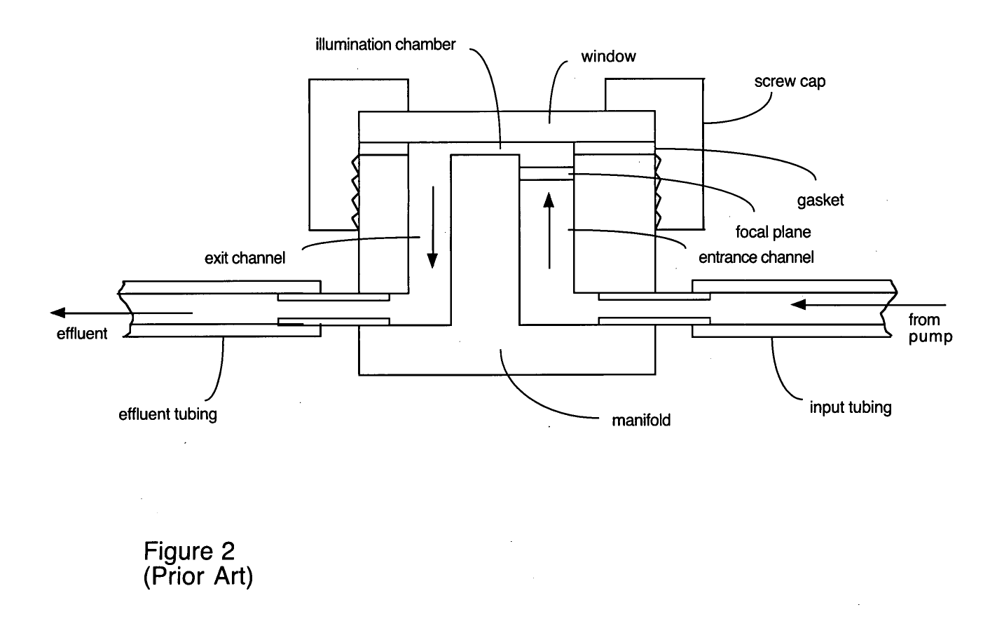

FIGS. 8A-B illustrate an improved flow cell for use in the separating apparatus. FIG. 8A is a plan view of flow cell 802. FIG. 8B is a front cutaway of flow cell 802. As mentioned above, better enumeration results from a design that reduces the amount of fluid diverted into specimen reservoir 430, while ensuring that a sufficient proportion of the target microorganisms are diverted into specimen 432. This is accomplished by minimizing the volume of fluid between detection of a particle and its sorting. Detection occurs at a focal plane (not shown) within the illuminated volume, in general in the upper part of channel 804 similar to FIG. 2 (prior art).

An important consideration in optimal sorting of particles in a Fountain Flow™ device is timing. In a perfect Fountain Flow™ Sorter the sorting mechanism would be positioned immediately after the portion of the flow cell in which fluorescent detections are made. This would allow for the sorting mechanism to be actuated immediately after detection for a short period of time, and then allowed to return to its default position.

However, in a real system there is a time delay, Δt, between the detection of a fluorescent cell and its passage through the sorting spout. Δt varies from particle to particle because the velocity of the fluid isn't constant across any tubing cross-section, but has a parabolic profile, with a maximum velocity along the axis of the tubing, approaching zero at the tubing walls. It can be shown that 75% of the particles flowing past a point in a cylindrical pipe travel at a velocity between the maximum velocity and 0.25× the maximum velocity. In order to sort particles correctly 75% of the time, the sorter solenoid must be activated (held in the positive sort position) over an appropriate time interval to accommodate particles traveling within this velocity range. This is called the activation time, T1 in FIG. 5.

We define the efficiency, Q, of our sorter as the total volume of sample divided by the volume of sample sorted for target cells, when 75% or greater of the cells are sorted correctly. If 1-ml of sample 404 is sorted so that 75% of the cells are in 10-μl of the positively sorted specimen 432 and 25% are in 990-μl of waste fluid, then Q=100. Using the Hagen-Poiseuille relationship one can show that to first order Q does not depend on the fluid flow velocity, but is inversely proportional to the (“dead”) volume between the location where a particle is optically detected and the sorting spout. In general, if one wants to sort rare particles (N per sample) into a subsample ˜1% of the sample volume, then the sorter tubing volume, between where the particle is optically detected and the spout 412, must be no larger than ˜1%/N of the total volume to be sampled. (In other words, a large dead volume leads to an inefficient sort.) For 1 particle/ml in 10-ml samples to be sorted into volumes of 10 μl, then the “dead” volume between detection and spout should also be no more than ˜10 μl.

Returning to FIGS. 8A and 8B, the miniaturized flow cell 802 shown reduces this volume of dead fluid substantially compared to prior art version of the fluid cell (shown in FIG. 2).

Flow cell 802 comprises a rectangular columnar body 810 with the illumination chamber 820, input channel 804, and output channel 806 on one end (the top in FIG. 8B). There is an oval-shaped O-ring grove 812, and cap 816 clamps window 818 against oval O-ring 814. Input channel 804 is connected to input tubing 826 which adheres 824 to a surface from which input channel 804 leads into body 810. Input channel 804 leads toward and up into chamber 820, and output channel 806 leads downward and away from chamber 820. Effluent piping 828 is connected to the outer end of output channel 806. When fluorescent detection device 408 makes use of flow cell 802, output tubing 828 connects to spout 412.

Illumination chamber 820 is formed between window 818, and the top of body 810. Oval O-ring 814 forms the walls of illumination chamber 820. Hence, input channel 804 leads into chamber 820 at point 805, and output channel 806 leads out of chamber 820 at point 807.

Cell 802 is functionally similar the prior art cell in FIG. 2, except for its dimensions and that it is not cylindrical.

Compared to the prior art flow cell of FIG. 2, the inside diameter of the channels 804, 806 has been reduced by a factor of 4 (from 4 mm to 1 mm), and the length of the channels by a factor of 3 (from 10 cm to 3 cm), for a reduction to 55 μl of dead volume. In addition, the volume of the illumination chamber 820 has been reduced by a similar factor. Thus, the dead space has been reduced by two orders of magnitude from ˜1.5-ml to ˜55 μl, as shown in Table 1. Hence, new flow cell 802 results in dead space of about 3% of the dead space in the larger flow cell.

TABLE 1
Comparison in the dimensions and dead space volume between the original
flow cell and the miniaturized flow cell shown in FIGS. 8A and 8B.

| | | | Total “dead” volume |
|---|---|---|---|
| | Approximate dimensions | Approx. dimensions of | including additional |
| | of illuminated volume | tubing 828 connected | volume between |
| | (cm) | to sorter spout (cm) | interior and exterior |
| Original Flow Cell | 1.6 × 0.4 × 0.4 ≈ 0.2 ml | 10 × 0.2 × 0.2 ≈ 1.2 ml | 1.5 ml |
| (4 mm orifice) | | | |
| New, miniature Flow | 3 × 0.05 × 0.05 ≈ 0.02 ml | 0.8 × 0.2 × 0.2 ≈ 0.025 ml | 0.055 ml |
| Cell (2 mm orifice) | | | |

FIG. 9 is a plot illustrating the expected results of the pro-sort detection using the present invention. This plot shows preliminary Fountain Flow™ cytometry results of the algae Procentrum minimum compared with conventional flow cytometry. Each point represents an FFC measurement of 17.5 ml of algae diluted in seawater to the concentrations shown here. Samples were measured with the FFC at a flow rate of 5 ml/min. The flow cell was illuminated with a green LED and imaged through a red longpass filter. (Illumination and emission wavelengths were not optimized for chlorophyll detection.)

Preliminary data using our provisional flow sorter prototype, is shown in Table 2 (Havens and Johnson, unpublished). This includes data taken sorting 90-micron Fluoresbrite fluorescent beads (PolySciences, Warrington PA) in distilled water. Because of the large “dead volume” between the flow cell and sorting spout each particle that is sorted is accompanied by ˜100 μl of fluid, several times larger than is possible with the miniaturized flow cell 802 of FIGS. 8A and 8B.

TABLE 2
Sorting data taken with Fountain Flow ™ Sorter
Prototype w 4 mm Orifice

| Target | Particle | Sample | Flow | Sorting |
|---|---|---|---|---|
| Particle | Concentration | Volume | Rate | Efficiency* |
| microbeads | 3.7/ml | 10 ml | 4 ml/min | 80 +/− 3% |
| microbeads | 88/ml | 190 ml | 30 ml/min | 90 +/− 3% |
| microbeads | 150/ml | 150 ml | 30 ml/min | 85 +/− 3% |

*Sorting efficiency here is the percentage of correct sorts when the sorter has a 2 s activation time.

It will be appreciated by one versed in the art that there are many possible variations on these designs within the scope of the present invention.

What is claimed is:

1. The method of detecting target microorganisms in an aqueous solution comprising the steps of:
(a) providing a sample of the solution containing fluorescent microorganisms, including fluorescent target microorganisms;
(b) flowing the sample through and out of a fluorescence detector;
(c) detecting fluorescent microorganisms with the fluorescence detector in the flowing sample;
(d) after detecting a fluorescent microorganism, diverting the flow out of the detector into a specimen reservoir for a period of time;
(e) after the period of time elapses, directing the flow out of the detector into a waste receptacle;
(f) introducing a selective stain chosen to tag the target microorganisms in the specimen reservoir; and
(g) detecting the tagged target microorganisms.

2. The method of claim 1 wherein step (a) is accomplished by introducing a primary stain into the sample, the primary stain selected to cause fluorescence of the microorganisms when illuminated by light at the appropriate wavelength(s).

3. The method of claim 1 wherein step (g) includes the steps of:
(g1) filtering the specimen to retain the target microorganisms on a filter; and
(g2) inspecting the filter with an epifluorescence microscope.

4. The method of claim 1 wherein step (g) comprises the steps of:
(g1) flowing the sample through a fluorescence detector; and
(g2) detecting target microorganisms with the detector.

5. The method of claim 1 wherein the target microorganisms comprise either unicellular or sub-cellular organisms.

6. The method of claim 5 wherein the target microorganisms belong to one of the following categories:
(a) bacteria;
(b) viruses;
(c) protozoa;
(d) algae;
(e) cyanobacteria;
or
(f) fungi.

7. The method of claim 1 wherein the step of choosing the selective stain comprises choosing either an RNA or a DNA labeling dye.

8. The method of claim 7, wherein the selective stain comprises one of the following:
(a) fluorescent in situ hybridization (FISH) dye(s); or
(b) fluorochrome-labeled antibodies to the target microorganisms.

9. The method of claim 1 wherein step (d) comprises the steps of:
(d1) receiving a detection signal;
(d2) pausing for a period T1; and
(d2) diverting the flow out of the detector into the specimen reservoir for a period of time T2;
wherein T1 and T2 are selected to result in diverting a majority of the target microorganisms into the specimen reservoir.

10. The method of claim 9 wherein T1 and T2 are further selected to result in a small fraction of the sample being diverted into the specimen reservoir.

11. The method of claim 10 wherein the small fraction constitutes about 1% of the sample.

12. Apparatus for detecting target microorganisms in an aqueous solution contain fluorescent target microorganisms and fluorescent non-target microorganisms comprising:
a fluorescence detection device for detecting fluorescent microorganisms in the sample as the sample flows through and out of the device;
a processor connected to the device for generating a flow control signal based upon fluorescence detected by the device;
a specimen reservoir;
a flow control element connected to the processor for diverting the sample flowing out of the device into the specimen reservoir based upon the control signal, the diverted sample forming a specimen; and
equipment for measuring target microorganisms in the specimen including a mechanism for tagging the target microorganisms and a mechanism for counting the tagged microorganisms.

13. The apparatus of claim 12, wherein the mechanism for counting comprises a tag detection device for detecting the tagged microorganisms as the specimen is flowed through the tag detection device and a processor connected to the tag detection device for counting the detected tagged microorganisms.

14. The apparatus of claim 13 wherein the tag detection device is the fluorescence detection device.

15. The apparatus of claim 12, wherein the mechanism for counting comprises a filter, a mechanism for filtering the specimen through the filter, and a fluorescent detection device for examining the filter for tagged microorganisms.

16. The apparatus of claim 12 wherein the fluorescence detection device comprises a flow cell having an input channel leading into the cell and upward, a detection window above the input channel, and an output channel leading away from the detection window and connected to the tube leading to the flow control device spout; and optics and electronics for detecting fluorescent particles through the detection window at a focal plane within the flow cell, wherein the sample flows into the input tube, across the cell adjacent to the window, and out the output tube.

17. The apparatus of claim 16 wherein the flow cell dimensions are selected to result in volume of under 0.05 ml between the focal plane and where the sample flows out the output tube.

18. The apparatus of claim 17 wherein the flow cell dimensions and the tube leading to the flow control device spout are selected to result in volume of under 0.2 ml between the focal plane and the flow control element spout.

19. Apparatus for detecting target microorganisms in a sample solution containing fluorescent microorganisms, including fluorescent target microorganisms, comprising:
a fluorescence detector which detects fluorescent microorganisms within a sample flowing through the detector;
means for diverting flow out of the detector into a specimen reservoir for a period of time after the detector detects a fluorescent microorganism and for directing the flow out of the detector into a waste receptacle after the period of time elapses;
means for diverting the flow out of the detector into a specimen reservoir for a period of time after the detector detects a fluorescent microorganism and for directing the flow out of the detector into a waste receptacle after the period of time elapses;
a mechanism for introducing a selective stain chosen to tag the target microorganisms into the specimen reservoir; and
means for detecting the tagged target microorganisms.
